(19) United States
(12) Patent Application Publication
(10) Pub. No.: US 2019/0381173 A1
(43) Pub. Date: Dec. 19, 2019

(54) METHODS FOR PHOTOBIOMODULATION OF BIOLOGICAL PROCESSES USING FLUORESCENCE GENERATED AND EMITTED FROM A BIOPHOTONIC COMPOSITION OR A BIOPHOTONIC SYSTEM

(71) Applicants: KLOX TECHNOLOGIES INC., Laval (CA); FB DERMATOLOGY LIMITED, Dublin (IE)

(72) Inventors: Michael CANOVA ENGELBRECHT NIELSEN, Frederiksberg C. (DK); Remigio PIERGALLINI, San Benedetto del Tronto (IT); Nikolaos LOUPIS, Athens (GR); Joanna JAWORSKA, Montreal (CA); Emmanuelle DEVEMY, Montreal (CA); Giovanni SCAPAGNINI, Catania (IT)

(21) Appl. No.: 16/480,691

(22) PCT Filed: Jan. 26, 2018
(86) PCT No.: PCT/CA2018/050099

§ 371 Date: Jul. 25, 2019

Related U.S. Application Data
(60) Provisional application No. 62/451,509, filed on Jan. 27, 2017.

(51) Int. Cl. A61K 41/00; C09K 11/07

(52) U.S. Cl. CPC A61K 41/0057; C09K 11/07

(57) ABSTRACT

According to various aspects, the present technology provides for the use of fluorescence generated and emitted from a biophotonic composition or system, wherein said fluorescence results from induction of one or more light-absorbing molecules found in the composition or system, wherein said emitted fluorescence is reaching a cell or tissue in order to modulate one or more biological processes within said cell or tissue.

### CROSS-REFERENCE TO RELATED APPLICATION

The present application claims priority to and benefit from U.S. Provisional Patent Application 62/451,509, filed on Jan. 27, 2017, the disclosure of which is incorporated herein by reference in its entirety.

### FIELD OF TECHNOLOGY

The present technology generally relates to methods for utilization of fluorescence generated through an induction of a biophotonic system. The fluorescence generated may be used to modulate cellular processes, including used for photobiomodulation of one or more biological processes in a cell or a tissue. The present technology also generally relates to methods for achieving photobiomodulation of one or more biological processes with fluorescence. The present technology further generally relates to methods for achieving photobiomodulation of one or more biological process using fluorescence generated and emitted from a photoactivated biophotonic system comprising one or more light-absorbing molecules.

### BACKGROUND INFORMATION

Light is a major source of energy that is used by organisms in a variety of biological processes, such as photosynthesis and vision, and it is sensed by specialized cells or other structures such as rods, cones and retinal ganglion cells, plastid and photoreceptor antennae. Biomedical research has recently shown that photons of light may be perceived in what has been traditionally thought of as non-photosensitive tissue and cells, for example the cells of the skin. Endogenous biological constituents, such as flavins, carotenoids and heme, are able to perceive photons and represent the photoreactive sites of larger photoreceptor molecules. Other photoreceptors include cytochrome c oxidase, cryptochromes, and opsin family proteins, which are widely expressed in different cells types.

The possibility of using visible light to trigger non-thermal, non-cytotoxic, biological reactions through photophysical events has been defined as photobiomodulation. Physiological and subsequent, therapeutic effects of photobiomodulation using incoherent light have been explored in several tissues and cell types.

In view of this, there is a need in the art to develop methods for inducing photobiomodulation of biological processes and systems wherein such photobiomodulation may be stimulated through application to a target cell or tissue of a light having a non-damaging, low level energy wherein said light is generated and emitted from a biophotonic composition or system that comprises one or more light-absorbing molecules being induced to generate and emit such light that may thus be used to modulate a biological process or system in a target cell or tissue.

### SUMMARY OF DISCLOSURE

According to various aspects, the present technology provides for the use of fluorescence generated and emitted from a biophotonic composition or system, wherein said fluorescence results from induction of one or more light-absorbing molecules found in the composition or system, wherein said emitted fluorescence is reaching a cell or tissue in order to modulate one or more biological processes within said cell or tissue.

According to various aspects, the present technology provides for the use of fluorescence generated and emitted from a biophotonic composition or system, wherein said fluorescence results from induction of one or more light-absorbing molecules found in the composition or system, wherein said emitted fluorescence is applied to a cell or tissue in order to modulate of a target biological process within said cell or tissue.

According to various aspects, the present technology provides for the use of fluorescence generated and emitted from a combination of light-absorbing molecules, wherein said emitted fluorescence is reaching a cell or tissue in order to modulate of a biological process within said cell or tissue.

According to various aspects, the present technology provides for a method of modulating a biological process in a target cell or tissue using fluorescence emitted from a biophotonic composition or system comprising one or more light-absorbing molecules. In some aspects, the method comprises identifying a biological process in a target cell or tissue to be modulated; applying a biophotonic composition or system comprising one or more light-absorbing molecules to the target cell or tissue; inducing emission of fluorescence having specific spectral emission properties from said one or more light-absorbing molecules; exposing said target cell or tissue to the emitted fluorescence having specific spectral properties; wherein said exposure of the target cell or tissue to the emitted fluorescence modulates the biological process in the target cell or tissue.

### BRIEF DESCRIPTION OF DRAWINGS

FIGS. 1A-1B are pictures of a system of multi-LED lights (“S-LED”) according to one embodiment of the present technology, built to have an emitted energy level(s) and an emission spectra that are the same or substantially the same as those generated as a result of an induction and emission of fluorescence from a biophotonic system comprising a multi-LED blue light lamp (“B-LED”) and a biophotonic composition comprising a light-absorbing molecule (referred to herein as “Biophotonic Composition A” or “BPC-A”).

FIG. 2 is a graph showing the spectral emission of a biophotonic system according to one embodiment of the present technology, upon illumination of BPC-A with the multi-LED blue light lamp (B-LED) versus the spectral emission from a system of multi-LEDs denoted as S-LED.

FIG. 3 is a graph showing total collagen production by Dermal Human Fibroblasts (“DHF”) (relative to the total collagen production of the control: non-treated DHF cells) following a treatment with either fluorescence emitted from the biophotonic composition BPC-A upon BCP-A being illuminated with the multi-LED blue light lamp B-LED to induce the generation of fluorescence by the light-absorbing molecule of BPC-A (second to left bar) in comparison to the total collagen production by DHFs upon their exposure to light emitted from the S-LED being (third to left bar). In a separate experiment, total collagen production by DHF cells that were exposed to light from the B-LED lamp was evaluated and the results are shown in the right-most bar.

FIG. 4 is a graph showing total collagen production by Dermal Human Fibroblasts as described for FIG. 3, with the DHF cells either not receiving an IFN-γ stimulation (left four bars) or receiving an IFN-γ stimulation (four right bars) in conjunction with their growth in culture and prior to being treated with either the fluorescence emitted from the BPC-A that has been induced by illumination with the B-LED lamp; or the being treated with the light emitted from the S-LED lamp; or following illumination with the B-LED alone.

FIG. 5A is a graph showing the cytotoxicity level measured by LDH activity in Dermal Human Fibroblasts upon the indicated treatment (no IFN-γ stimulation).

FIG. 5B is a graph showing the cytotoxicity level measured by LDH activity in Dermal Human Fibroblasts upon the indicated treatment (with or without IFN-γ stimulation).

FIG. 6 is a graph showing the level of TNF-α mRNA total expression in DHF treated as indicated. Continues: 2 min illumination; Fractionated: 1 minute illumination followed by 1 minute break followed by 1 minute illumination.

FIG. 7A is a schematic representation of the experimental set defined in Example 4.

FIG. 7B panel A, panel B, panel C and panel D are pictures of Human Aortic Endothelial cells (HAECs) subjected to the indicated treatments: Non-treated CTRL (top left panel); Positive CTRL (VEGF 30 ng/ml) (top right panel) treated by fluorescence emitted from BPC-B (bottom left panel); and light from multi-LED lamp only (bottom right panel).

FIG. 7C is a graph showing the effect of conditioned medium derived from fluorescent treated DHF on tube formation of human aortic endothelial cells.

### DESCRIPTION OF TECHNOLOGY

The present technology is explained in greater detail below. This description is not intended to be a detailed catalog of all the different ways in which the technology may be implemented, or all the features that may be added to the instant technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant technology. Hence, the following specification is intended to illustrate some particular embodiments of the technology, and not to exhaustively specify all permutations, combinations and variations thereof.

As used herein, the singular form “a,” “an” and “the” include plural referents unless the context clearly dictates otherwise.

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “biophotonic” means the generation, manipulation, detection and application of photons in a biologically relevant context. In other words, biophotonic compositions exert their physiological effects primarily due to the generation and manipulation of photons. “Biophotonic composition” is a composition as described herein that may be activated by light to produce photons for biologically relevant applications.

The present technology stems from studies performed by the Inventors aimed at comparing the effects on modulation of biological processes of the following light emitting systems:
i) a fluorescent light emitted by a biophotonic composition or system as defined herein and comprising one or more light-absorbing molecules. Wherein, upon being illuminated by a light source (e.g., a multi-LED blue light lamp having a wavelength range and an energetic capacity to induce excitation of the one or more light-absorbing molecules), the one or more light-absorbing molecules release energy in the form of fluorescence, which fluorescence is subsequently emitted from the biophotonic composition or system; with
ii) a non-fluorescent light emitted by a light source designed and constructed to have an illumination output that mimics or encompasses the same energy and emission spectra as the fluorescence emitted from the biophotonic composition or system. Such a source of non-fluorescent light was constructed from a system of light emitting diodes (LED) and is referred herein as “S-LED light source”.

Surprisingly, the Inventors have found that fluorescence generated from induction (photoactivation) of one or more light-absorbing molecules is more efficient than a non-fluorescent light emitted by an artificial light source in modulating specific biological processes even if the non-fluorescent light emitted by the artificial S-LED light source possesses some of the properties of the fluorescence emitted by the one or more light-absorbing molecules.

These findings by the Inventors indicate that fluorescence emitted from an induced (photoactivated) biophotonic composition or system has certain properties that are important for modulation of biological processes.

As used herein, the term “photobiomodulation” refers to form of light therapy that utilizes nonionizing forms of light sources, including lasers, light-emitting diodes (LED), and broadband light, in the visible and infrared spectrum. It is a nonthermal process involving endogenous light-absorbing molecules eliciting photophysical (i.e., linear and nonlinear) and photochemical events at various biological scales.

As used herein, the expressions “light-absorbing molecule”, “photoactivatable agent”, “photoactivating agent” and “chromophore” are used herein interchangeably and mean a molecule, when contacted by light irradiation, is capable of absorbing the light. The light-absorbing molecule readily undergoes photoexcitation and can then transfer its energy to other molecules or emit it as light. The light-absorbing molecule may be a synthetic light-absorbing molecule, such as a small molecule, or may be a naturally-occurring light-absorbing molecule that may also be a small molecule or a biological molecule or a sub-unit thereof, such as a protein or peptide or a subunit thereof comprising a sequence of amino acids shorter than a peptide.

In some embodiments, the light-absorbing molecule of the present technology absorbs at a wavelength in the range of the visible spectrum, such as at a wavelength of from about 380 to about 800 nm, such as from about 380 to about 700 nm, or from about 380 to about 600 nm.

In some embodiments, the light-absorbing molecule absorbs at a wavelength of from about 200 nm to about 800 nm, such as from about 200 nm to about 700 nm, from about 200 nm to about 600 nm, or from about 200 nm to about 500 nm.

In some embodiments, the light-absorbing molecule absorbs at a wavelength of from about 200 nm to about 600 nm.

In some embodiments, the light-absorbing molecule absorbs light at a wavelength of from about 200 nm to about 300 nm, from about 250 nm to about 350 nm, from about 300 nm to about 400 nm, from about 350 nm to about 450 nm, from about 400 nm to about 500 nm, from about 400 nm to about 600 nm, from about 450 nm to about 650 nm, from about 600 nm to about 700 nm, from about 650 nm to about 750 nm, or from about 700 nm to about 800 nm.

In some embodiments, the light-absorbing molecule of the present technology undergoes partial or complete photobleaching upon application of light. By photobleaching is meant a photochemical destruction of the light-absorbing molecule, which can generally be characterized as a visual loss of color or loss of fluorescence. It will be appreciated to those skilled in the art that optical properties of a particular light-absorbing molecule may vary depending on the light-absorbing molecule's surrounding medium.

In some instances, the combination of different light-absorbing molecules may increase photoabsorption by the combined light-absorbing molecule molecules and enhance absorption and photobiomodulation selectivity. This creates multiple possibilities of generating new photosensitive, and/or selective light-absorbing molecules mixtures for use in the context of the present technology.

In some embodiments, the light-absorbing molecule(s) comprising the biophotonic composition or system of the present technology is/are selected such that their emitted fluorescent light, on photoactivation or photobiomodulation, is within one or more of the green, yellow, orange, red and infrared portions of the electromagnetic spectrum, for example having a peak wavelength within the range of about 490 nm to about 800 nm.

In some embodiments, the fluorescence emitted from the biophotonic composition or system of the present technology has a power density of between 0.005 mW/cm² to about 10 mW/cm² or about 0.5 mW/cm² to about 5 mW/cm².

Examples of light-absorbing molecules that may in part comprise the biophotonic composition or system of the present technology include xanthene derivatives, azo dyes, biological stains, carotenoids and chlorophyll dyes. The xanthene group consists of three sub-groups: a) the fluorenes; b) fluorones; and c) the rhodoles. The fluorenes group comprises the pyronines (e.g., pyronine Y and B) and the rhodamines (e.g., rhodamine B, G and WT). The fluorone group comprises the fluorescein dye and the fluorescein derivatives. Fluorescein is a fluorophore commonly used in microscopy with an absorption maximum of about 494 nm and an emission maximum of about 521 nm. The disodium salt of fluorescein is known as D&C Yellow 8.

Further examples of light-absorbing molecules that may in part comprise the biophotonic composition or system of the present technology include the eosins group of molecules. Eosin Y (tetrabromofluorescein, acid red 87, D&C Red 22), a fluorescent compound with an absorption maximum of from about 514 to about 518 nm that stains the cytoplasm of cells, collagen, muscle fibers and red blood cells intensely red; and Eosin B (acid red 91, eosin scarlet, dibromo-dinitrofluorescein), with the same staining characteristics as Eosin Y. Eosin Y and Eosin B are collectively referred to as “Eosin,” and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light. Phloxine B (2,4,5,7 tetrabromo 4,5,6,7,tetrachlorofluorescein, D&C Red 28, acid red 92) is a red dye derivative of fluorescein which is used for disinfection and detoxification of waste water through photooxidation. It has an absorption maximum of 535-548 nm.

Erythrosine B, or simply Erythrosine or Erythrosin (acid red 51, tetraiodofluorescein) is a cherry-pink, coal-based fluorine food dye with a maximum absorbance of 524-530 nm in aqueous solution. It is subject to photodegradation.

Rose Bengal (4,5,6,7 tetrachloro 2,4,5,7 tetraiodofluorescein, acid red 94) is a bright bluish-pink fluorescein derivative with an absorption maximum of 544-549 nm.

Merbromine (mercurochrome) is an organo-mercuric disodium salt of fluorescein with an absorption maximum of 508 nm.

The azo (or diazo-) dyes share the N—N group, called azo the group and include methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

Biological stains include, but not limited to: saffranin (Saffranin 0, basic red 2) is an azo-dye, fuchsin (basic or acid) (rosaniline hydrochloride) is a magenta biological dye having an absorption maximum of 540-555 nm; 3,3′-dihexylocarbocyanine iodide (DiOC6), carminic acid (acid red 4, natural red 4), indocyanin green (ICG).

Carotenoid dyes include saffron red powder. Saffron contains more than 150 different compounds, many of which are carotenoids: mangicrocin, reaxanthine, lycopene, and various α and β-carotenes.

Examples of chlorophyll dyes include but are not limited to chlorophyll a, chlorophyll b, oil soluble chlorophyll, bacteriochlorophyll a, bacteriochlorophyll b, bacteriochlorophyll c, bacteriochlorophyll d, protochlorophyll, protochlorophyll a, amphiphilic chlorophyll derivative 1, and amphiphilic chlorophyll derivative 2.

Further examples of light-absorbing molecules that may, in part, comprise the biophotonic composition or system of the present technology: Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Saffranin O), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Celestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In some embodiments, the light created by emission of fluorescent light from light-absorbing molecules having a photobiomodulatory effects has low energy as outlined in Table 1.

TABLE 1
Examples of Energy of fluorescence emitted
from light-absorbing molecules

| | | |
|---|---|---|
| Time (min) | Φ_{400 nm to 540} [W/m²] | Φ_{540 nm to 660} [W/m²] |

| | | | |
|---|---|---|---|
| 0 | min | 40.8 | 0.150 |
| 5 | min | 48.0 | 0.119 |
| 10 | min | 57.2 | 0.098 |
| 15 | min | 63.7 | 0.079 |
| 18 | min | 65.6 | 0.073 |

With respect to light-absorbing molecules that may, in part, comprise the biophotonic composition or system of the present technology, wherein such light-absorbing molecules are derived from a naturally-occurring source and hence the light-absorbing molecule(s) may be referred to as a “natural light-absorbing molecule”, such sources of light-absorbing molecules include, but are not limited to, a plant source, an animal source, an amphibian source, a fungal source, an algal source, a marine or terrestrial microorganism source, or a marine or terrestrial invertebrate source.

In some embodiments of the present technology, with respect to a plant-derived light-absorbing molecule, the plant-derived light-absorbing molecule is obtained from a plant extract, for example, but not limited to, extracts of coffee beans, green tea leaves, blueberries, cranberries, huckleberries, acai berries, goji berries, blackberries, raspberries, grapes, strawberries, persimmon, pomegranate, lingonberry, bearberry, mulberry, bilberry, choke cherry, sea buckthorn berries, goji berry, tart cherry, kiwi, plum, apricot, apple, banana, berry, blackberry, blueberry, cherry, cranberry, currant, greengage, grape, grapefruit, gooseberry, lemon, mandarin, melon, orange, pear, peach, pineapple, plum, raspberry, strawberry, sweet cherry, watermelon, and wild strawberry. In some embodiments, the plant-derived light-absorbing molecule is obtained from trees, including for instance sequoia, coastal redwood, bristlecone pine, birch, and cedar.

In other embodiments, the plant-derived light-absorbing molecule is obtained from leafy or salad vegetables [e.g., Amaranth (Amaranthus cruentus), Arugula (Eruca sativa), Beet greens (Beta vulgaris subsp. vulgaris), Bitterleaf (Vernonia calvoana), Bok choy (Brassica rapa Chinensis group), Broccoli Rabe (Brassica rapa subsp. rapa), Brussels sprout (Brassica oleracea Gemmifera group), Cabbage (Brassica oleracea Capitata group), Catsear (Hypochaeris radicata), Celery (Apium graveolens), Celtuce (Lactuca sativa var. asparagine), Ceylon spinach (Basella alba), Chard (Beta vulgaris var. cicla), Chaya (Cnidoscolus aconitifolius subsp. aconitifolius), Chickweed (Stellaria), Chicory (Cichorium intybus), Chinese cabbage (Brassica rapa Pekinensis group), Chinese Mallow (Malva verticillata), Chrysanthemum leaves (Chrysanthemum coronarium), Collard greens (Brassica oleracea), Corn salad (Valerianella locusta), Cress (Lepidium sativum), Dandelion (Taraxacum officinale), Endive (Cichorium endivia), Epazote (Chenopodium ambrosioides), Fat hen (Chenopodium album), Fiddlehead (Pteridium aquilinum, Athyrium esculentum), Fluted pumpkin (Telfairia occidentalis), Garden Rocket (Eruca sativa), Golden samphire (Inula crithmoides), Good King Henry (Chenopodium bonus-henricus), Greater Plantain (Plantago major), Kai-lan (Brassica rapa Alboglabra group), Kale (Brassica oleracea Acephala group), Komatsuna (Brassica rapa Pervidis or Komatsuna group), Kuka (Adansonia spp.), Lagos bologi (Talinum fruticosum), Land cress (Barbarea verna), Lettuce (Lactuca sativa), Lizard's tail (Houttuynia cordata), Melokhia (Corchorus olitorius, Corchorus capsularis), Mizuna greens (Brassica rapa Nipposinica group), Mustard (Sinapis alba), New Zealand Spinach (Tetragonia tetragonioides), Orache (Atriplex hortensis), Paracress (Acmella oleracea), Pea sprouts/leaves (Pisum sativum), Polk (Phytolacca americana), Radicchio (Cichorium intybus), Samphire (Crithmum maritimum), Sea beet (Beta vulgaris subsp. maritima), Seakale (Crambe maritima), Sierra Leone bologi (Crassocephalum spp.), Soko (Celosia argentea), Sorrel (Rumex acetosa), Spinach (Spinacia oleracea), Summer purslane (Portulaca oleracea), Swiss chard (Beta vulgaris subsp. cicla var. flavescens), Tatsoi (Brassica rapa Rosularis group), Turnip greens (Brassica rapa Rapifera group), Watercress (Nasturtium officinale), Water spinach (Ipomoea aquatica), Winter purslane (Claytonia perfoliata), Yarrow (Achillea millefolium)]; fruiting and flowering vegetables, such as those from trees [e.g., Avocado (Persea americana), Breadfruit (Artocarpus altilis)]; or from annual or perennial plants [e.g., Acorn squash (Cucurbita pepo), Armenian cucumber (Cucumis melo Flexuosus group), Aubergine (Solanum melongena), Bell pepper (Capsicum annuum), Bitter melon (Momordica charantia), Caigua (Cyclanthera pedata), Cape Gooseberry (Physalis peruviana), Capsicum (Capsicum annuum), Cayenne pepper (Capsicum frutescens), Chayote (Sechium edule), Chili pepper (Capsicum annuum Longum group), Courgette (Cucurbita pepo), Cucumber (Cucumis sativus), Eggplant (Solanum melongena), Luffa (Luffa acutangula, Luffa aegyptiaca), Malabar gourd (Cucurbita ficifolia), Parwal (Trichosanthes dioica), Pattypan squash (Cucurbita pepo), Perennial cucumber (Coccinia grandis), Pumpkin (Cucurbita maxima, Cucurbita pepo), Snake gourd (Trichosanthes cucumerina), Squash aka marrow (Cucurbita pepo), Sweet corn aka corn; aka maize (Zea mays), Sweet pepper (Capsicum annuum Grossum group), Tinda (Praecitrullus fistulosus), Tomatillo (Physalis philadelphica), Tomato (Lycopersicon esculentum var), Winter melon (Benincasa hispida), West Indian gherkin (Cucumis anguria), Zucchini (Cucurbita pepo)]; the flower buds of perennial or annual plants [e.g., Artichoke (Cynara cardunculus, C. scolymus), Broccoli (Brassica oleracea), Cauliflower (Brassica oleracea), Squash blossoms (Cucurbita spp.); podded vegetables [e.g., American groundnut (Apios americana), Azuki bean (Vigna angularis), Black-eyed pea (Vigna unguiculata subsp. unguiculata), Chickpea (Cicer arietinum), Common bean (Phaseolus vulgaris), Drumstick (Moringa oleifera), Dolichos bean (Lablab purpureus), Fava bean (Vicia faba), Green bean (Phaseolus vulgaris), Guar (Cyamopsis tetragonoloba), Horse gram (Macrotyloma uniflorum), Indian pea (Lathyrus sativus), Lentil (Lens culinaris), Lima Bean (Phaseolus lunatus), Moth bean (Vigna acontifolia), Mung bean (Vigna radiata), Okra (Abelmoschus esculentus), Pea (Pisum sativum), Peanut (Arachis hypogaea), Pigeon pea (Cajanus cajan), Ricebean (Vigna umbellata), Runner bean (Phaseolus coccineus), Soybean (Glycine max), Tarwi (tarhui, chocho; Lupinus mutabilis), Tepary bean (Phaseolus acutifolius), Urad bean (Vigna mungo), Velvet bean (Mucuna pruriens), Winged bean (Psophocarpus tetragonolobus), Yardlong bean (Vigna unguiculata subsp. sesquipedalis)]; bulb and stem vegetables [e.g., Asparagus (Asparagus officinalis), Cardoon (Cynara cardunculus), Celeriac (Apium graveolens var. rapaceum), Celery (Apium graveolens), Elephant Garlic (Allium ampeloprasum var. ampeloprasum), Florence fennel (Foeniculum vulgare var. dulce), Garlic (Allium sativum), Kohlrabi (Brassica oleracea Gongylodes group), Kurrat (Allium ampeloprasum var. kurrat), Leek (Allium porrum), Lotus root (Nelumbo nucifera), Nopal (Opuntia ficus-indica), Onion (Allium cepa), Prussian asparagus (Ornithogalum pyrenaicum), Shallot (Allium cepa Aggregatum group), Welsh onion (Allium fistulosum), Wild leek (Allium tricoccum)]; root and tuberous vegetables [e.g., Ahipa (Pachyrhizus ahipa), Arracacha (Arracacia xanthorrhiza), Bamboo shoot (Bambusa vulgaris and Phyllostachys edulis), Beetroot (Beta vulgaris subsp. vulgaris), Black cumin (Bunium persicum), Burdock (Arctium lappa), Broadleaf arrowhead (Sagittaria latifolia), Camas (Camassia), Canna (Canna spp.), Carrot (Daucus carota), Cassava (Manihot esculenta), Chinese artichoke (Stachys affinis), Daikon (Raphanus sativus Longipinnatus group), Earthnut pea (Lathyrus tuberosus), Elephant Foot yam (Amorphophallus paeoniifolius), Ensete (Ensete ventricosum), Ginger (Zingiber officinale), Gobo (Arctium lappa), Hamburg parsley (Petroselinum crispum var. tuberosum), Jerusalem artichoke (Helianthus tuberosus), Emma (Pachyrhizus erosus), Parsnip (Pastinaca sativa), Pignut (Conopodium majus), Plectranthus (Plectranthus spp.), Potato (Solanum tuberosum), Prairie turnip (Psoralea esculenta), Radish (Raphanus sativus), Rutabaga (Brassica napus Napobrassica group), Salsify (Tragopogon porrifolius), Scorzonera (Scorzonera hispanica), Skirret (Sium sisarum), Sweet Potato or Kumara (Ipomoea batatas), Taro (Colocasia esculenta), Ti (Cordyline fruticosa), Tigernut (Cyperus esculentus), Turnip (Brassica rapa Rapifera group), Ulluco (Ullucus tuberosus), Wasabi (Wasabia japonica), Water chestnut (Eleocharis dulcis), Yacon (Smallanthus sonchifolius), Yam (Dioscorea spp.)]; spices and other flavorings [e.g., ajowan (Trachyspermum ammi) allspice (Pimenta dioica), amchur (Mangifera indica), angelica (Angelica spp.), anise (Pimpinella anisum), annatto (Bixa orellana), asafoetida (Ferula asafoetida), barberry (Berberis spp (many) and Mahonia spp (many)), basil (Ocimum spp)., bay leaf (Laurus nobilis), bee balm (bergamot, monarda; Monarda spp.), black cumin (Bunium persicum), black lime (loomi; Citrus aurantifolia), boldo (boldina; Peumus boldus), bush tomato (akudjura; Solanum central), borage (Borago officinalis), calamus (sweet flag; Acorus calamus), candlenut (Aleurites moluccana), caraway (Carum carvi), cardamom (Amomum compactum), capers (Capparis spinosa), cassia (Cimmanmomum cassia), cayenne pepper (Capsicum annum), celery (Apium graveolens), chervil (Anthriscus cerefolium), chicory (Cicorium intybus), chile/chili/chilli (e.g., Capsicum frutescens), chile varieties (Capsicum frutescens), chives (Allium odorum, Allium shoenoprasum), cilantro (Coriandrum sativum), cinnamon (Cinnamomum zeylanicum; Cinnamomum cassia), clove (Syzygium aromaticum), coriander (Coriandrum sativum), cubeb (Piper cubeba), cumin (Cuminum cyminum), curry leaf (kari; Murraya koenigii), dill (Anethum graveolens), elder (elder flower, & elderberry; Sambucus nigra), epazote (Chenopodium ambrosioides), fennel (Foeniculum vulgare), fenugreek (Trigonella foenum-graecum), galangal (Alpinia galangal), garlic (Allium sativum), ginger (Zingiber officinale), hoja santa (Piper auritum), horseradish (Armoracia rusticana), hyssop (Hyssopus officinalis), jamaican sorrel (Hibiscus sabdariffa), juniper (Juniperus communis), kaffir lime (Citrus hystrix), mustard (Brassica nigra), kokum (Garcinia indica), lavender (Lavandula angustifolia), lemon balm (Melissa officinalis), lemon grass (Cymbopogon citrates), lemon myrtle (Backhousia citriodora), lemon verbena (Lippia citriodora), licorice (Glycyrrhiza glabra), lovage (Levisticum officinale), mace (Myristica fragrans), mahlab (Prunus mahaleb), marjoram (Majorana hortensis), mastic (Pistacia lenticus), melegueta pepper (Aframomum melegueta), grains of paradise (Aframomum granum paradise), mint (Mentha spp.), mountain pepper (Tasmannia lanceolata), Tasmanian pepper (Tasmannia lanceolata), myrtle (Myrtus communis), nigella (Nigella sativa), nutmeg (Myristica fragrans), onion (Allium cepa), orris root (Germanica florentina), paprika (Capsicum annuum), parsley (Petroselinum crispum), pepper (Piper nigrum), poppy seed (Papaver somniferum), rosemary (Rosmarinus officinalis), saffron (Crocus sativus), sage (Salvia officinalis), sassafras (Sassafras albidum), savory (Satureja hortensis), scented geranium (Pelargonium spp), screw-pine (pandan; Pandanus tectorius), sesame (Sesamum indicum), soapwort (Saponaria officinalis), sorrel (Rumex acetosa), star anise (Illicium verum), sumac (Rhus coriaria), szechwan pepper (Zanthoxylum spp. (piperitum, simulans, bungeanum, rhetsa acanthopodium)), tamarind (Tamarindus indica), tarragon (Artemisia dracunculus), thyme (Thymus vulgaris), turmeric (Curcuma longa), vanilla (Vanilla planifolia), wasabi (Wasabia japonica), watercress (Nasturtium officinale), wattleseed (Acacia aneuro), zedoary (Curcuma zedoaria), and sea vegetables [e.g., Aonori (Monostroma spp., Enteromorpha spp.), Carola (Callophyllis variegate), Dabberlocks aka badderlocks (Alaria esculenta), Dulse (Palmaria palmata), Gim (Porphyra spp.), Hijiki (Hizikia fusiformis), Kombu (Laminaria japonica), layer (Porphyra spp.), Mozuku (Cladosiphon okamuranus), Nori (Porphyra spp.), Ogonori (Gracilaria spp.), Sea grape (Caulerpa spp.), Seakale (Crambe maritima), Sea lettuce (Ulva lactuca), Wakame (Undaria pinnatifida)], some of which are not plants in the taxonomic sense.

Light-absorbing molecules that may, in part, comprise the biophotonic composition or system of the present technology may be selected, for example, on their emission wavelength properties or on the basis of their energy transfer potential, or other properties the that specific light-absorbing molecule may exhibit apart from its capacity to absorb incident light and emit fluorescence.

As used herein, the expression “properties of the fluorescence emitted from the chrompohore” includes, but is not limited to, one or more of emission spectra, wavelength of the emitted fluorescence, radiant fluency of the emitted fluorescence, power density of the emitted fluorescence, fluorescence excitation spectrum, absorption spectrum, fluorescence emission spectrum, extinction coefficient, fluorescence quantum yield (QY), quenching and photobleaching.

As used herein, the expressions “a biological process” and “biological processes” refers to processes and cellular or biological pathways or networks that may be required for the proper functionality of a living organism, cell or tissue or that are required so as to enable a cell or tissue to respond to an external or internal stimulatory event or to respond to a change in its environment or to produce a specific biological compound as a result of stimulatory event's reception by the cell, tissue or organism. Biological processes are made up of many chemical reactions or other events that are involved in the persistence and transformation of life forms. Metabolism and homeostasis are examples of biological processes. Modulation of biological processes occurs when any biological process is modulated in its frequency, rate or extent. Biological processes are regulated by many means, such as, for example, control of gene expression, protein modification or interaction with a protein or substrate molecule.

Other biological processes include, but are not limited to, physiological process (i.e., those processes specifically pertinent to the functioning of integrated living units: cells, tissues, organs, limbs, and organisms); reproductive processes; digestive processes; response to stimulus (e.g., a change in state or activity of a cell or an organism (in terms of movement, secretion, enzyme production, gene expression, or the like.) as a result of a stimulus); interaction between organisms (i.e., the processes by which an organism has an observable effect on another organism of the same or different specie); cell growth; cellular differentiation; fermentation; fertilisation; germination; tropism; hybridisation; metamorphosis; morphogenesis; photosynthesis; and transpiration.

In some aspects of the present technology, biological processes also include cellular processes. As used herein, the expression “cellular processes” refers to processes that are carried out at the cellular level, but are not necessarily restricted to a single cell. For example, cell communication occurs among more than one cell, but occurs at the cellular level.

Examples of cellular processes include, but are not limited to, cell communication, cellular senescence, DNA repair, gene expression, meiosis, metabolism, necrosis, nuclear organization, programmed cell death, and protein targeting.

Other examples of cellular processes include: actin nucleation core, action potential, afterhyperpolarization, apoptosis, autolysis (biology), autophagin, autophagy, cell cycle, branch migration, bulk endocytosis, cap formation, CDK7 pathway, cell death, cell division, cell division orientation, cell growth, cell migration, cellular differentiation, cellular senescence, cell signaling (e.g, intracrine signaling, autocrine signaling, juxtacrine signaling, paracrine signaling, endocrine signaling), chromatolysis, chromosomal crossover, coagulative necrosis, cytoplasm-to-vacuole targeting, cytoplasmic streaming, cytostasis, centinogenesis, DNA repair, efferocytosis, emperipolesis, endocytic cycle, endocytosis, endoexocytosis, endoplasmic-reticulum-associated protein degradation, epithelial-mesenchymal transition, Exocytosis, fibrinoid necrosis, filamentation, formins, genetic recombination, histone methylation, induced pluripotent stem-cell therapy, interference (genetic), interkinesis, intracellular transport, intraflagellar transport, invagination, karyolysis, karyorrhexis, klerokinesis, leptotene stage, meiosis, membrane potential, microautophagy, necrobiology, necrobiosis, necroptosis, necrosis, nemosis, nuclear organization, parasexual cycle, parthanatos, passive transport, peripolesis, phagocytosis, phagoptosis, pinocytosis, poly(adp-ribose) polymerase family member 14, potocytosis, pyknosis, quantal neurotransmitter release, Rap6, receptor-mediated endocytosis, residual body, Ribosome biogenesis, senescence, septin, Site-specific recombination, squelching, trans-endocytosis, transcytosis, xenophagy.

In some aspects, the cellular processes are cellular signaling processes. As used herein, the expression “cellular signaling processes” refers to the process by which a chemical or physical signal is transmitted through a cell as a series of molecular events, most commonly protein phosphorylation, which ultimately result in a response. Proteins responsible for detecting stimuli are generally termed receptors, although in some cases the term sensor is used. The changes elicited by ligand binding (or signal sensing) in a receptor give rise to a cascade of biochemical events along a signaling pathway. When signaling pathways interact with one another they form networks, which allow cellular responses to be coordinated. At the molecular level, such responses include changes in the transcription or translation of genes, and post-translational and conformational changes in proteins, as well as changes in their location. These molecular events are the basic mechanisms controlling cell growth, proliferation, metabolism and many other processes.

In some aspects of these embodiments, “cellular processes” includes physical properties of a cell or group of cells such a, but not limited to, atomic forces, molecular vibration, resonance, orientation, and energy transfer level which mat be modulated by the methods of the present technology.

In some embodiments, the present technology provides a method for modulating a biological process in a subject using artificially created fluorescence. In some aspects of this embodiment, the artificially created fluorescence shares substantially all of the same properties of a naturally created fluorescence, which properties are required to modulate a biological process. In some instances, the method further requires observing the effects of fluorescence emitted from a fluorescent compound or a combination of fluorescent compounds on a specific biological process. In some implementations, this step may be accomplished by using a biophotonic system to create the naturally created fluorescence.

As used herein, the expressions “biophotonic composition” and “biophotonic system” refers to biophotonic compositions and systems that are comprised of, in part, a light-absorbing molecule that may be induced into an excited state as a result of the light-absorbing molecule's being illuminated by light (e.g., photons) of a specific wavelength and thereafter releasing fluorescence, wherein the fluorescence is emitted from the biophotonic composition or system. These biophotonic compositions contain at least one light-absorbing molecule that may be activated by light and accelerates the dispersion of fluorescence light energy from the biophotonic composition, which leads to the emitted fluorescence having a modulating effect on its own to a biological process or biological processes in a target cell or tissue.

In certain aspects, the biophotonic compositions of the present technology are substantially transparent/translucent and/or have high light transmittance in order to permit light dissipation into and through the biophotonic composition. In this way, the area of the target tissue under the composition or the target cells to which the biophonic composition may be applied can be treated both with the fluorescent light emitted by the composition and the light irradiating the composition to activate it, which may benefit from the different therapeutic effects of light having different wavelengths.

The biophotonic composition can be in the form of a semi-solid or viscous liquid, such as a gel, or are gel-like, and which have a spreadable consistency at room temperature (e.g., about 20-25° C.), prior to illumination. By spreadable is meant that the composition can be topically applied to a treatment site at a thickness of less than about 0.5 mm, from about 0.5 mm to about 3 mm, from about 0.5 mm to about 2.5 mm, or from about 1 mm to about 2 mm

In some embodiments, the biophotonic compositions of the present technology comprise oxidants as a source of oxygen radicals.

In some embodiments, the light-absorbing molecule or combination of light-absorbing molecules is present in an amount of from about 0.001% to about 40% by weight of the total composition. In some embodiments, the light-absorbing molecule or combination of light-absorbing molecules is present in an amount of from about 0.005% to about 2%, from about 0.01% to about 1%, from about 0.01% to about 2%, from about 0.05% to about 1%, from about 0.05% to about 2%, from about 0.1% to about 1%, from about 0.1% to about 2%, from about 1-5%, from about 2.5% to about 7.5%, from about 5% to about 10%, from about 7.5% to about 12.5%, from about 10% to about 15%, from about 12.5% to about 17.5%, from about 15% to 20%, from about 17.5% to about 22.5%, from about 20% to about 25%, from about 22.5% to about 27.5%, from about 25% to about 30%, from about 27.5% to about 32.5%, from about 30% to about 35%, from about 32.5% to about 37.5%, or from about 35% to about 40% by weight of the total composition. In some embodiments, the light-absorbing molecule or combination of light-absorbing molecules is present in an amount of at least about 0.2% by weight of the total composition.

In some embodiments, the light-absorbing molecule or combination of light-absorbing molecules is present in an amount of 0.001% to 40% by weight of the total composition. In some embodiments, the light-absorbing molecule or combination of light-absorbing molecules is present in an amount of from 0.005 to 2%, from 0.01 to 1%, from 0.01% to 2%, from 0.05% to 1%, from 0.05-2%, from 0.1% to 1%, from 0.1% to 2%, from 1 to 5%, from 2.5 to 7.5%, from 5 to 10%, from 7.5% to 12.5%, from 10% to 15%, from 12.5% to 17.5%, from 15% to 20%, from 17.5% to 22.5%, from 20% to 25%, from 22.5% to 27.5%, from 25% to 30%, from 27.5% to 32.5%, from 30% to 35%, from 32.5% to 37.5%, or from 35% to 40% by weight of the total composition. In some embodiments, the light-absorbing molecule or combination of light-absorbing molecules is present in an amount of at least 0.2% by weight of the total composition.

The method of the present technology further comprises a step of identification of the properties of a fluorescence emitted by an excited light-absorbing molecule of the biophotonic composition that thereby allows for a modulation of a biological process in the target cell or tissue wherein the target cell or tissue is exposed to the fluorescence emitted by the induced biophotonic composition. The method also comprises exposing the target cell or tissue to the fluorescence emitted from the induced biophonic composition, whereby exposure of the target cell of tissue to the emitted fluorescence modulates a biological process in the target cell or tissue.

Identification of equivalent compositions, methods and kits are well within the skill of the ordinary practitioner and would require no more than routine experimentation, in light of the teachings of the present technology. Practice of the disclosure will be still more fully understood from the following examples, which are presented herein for illustration only and should not be construed as limiting the disclosure in any way.

The present technology is illustrated in the following non-limiting examples.

### EXAMPLES

The examples below are given so as to illustrate the practice of various embodiments of the present technology. They are not intended to limit or define the entire scope of this technology. It should be appreciated that the technology is not limited to the particular embodiments described and illustrated herein but includes all modifications and variations falling within the scope of the disclosure as defined in the appended embodiments.

### Example 1

### S-LED Light Setup for Testing Light Emitted by S-LED vs. Fluorescence Emitted by a Biophotonic Composition Comprising One or More Light-Absorbing Molecules that have been Induced to qn Excited State to Modulate pn Photobiomodulation

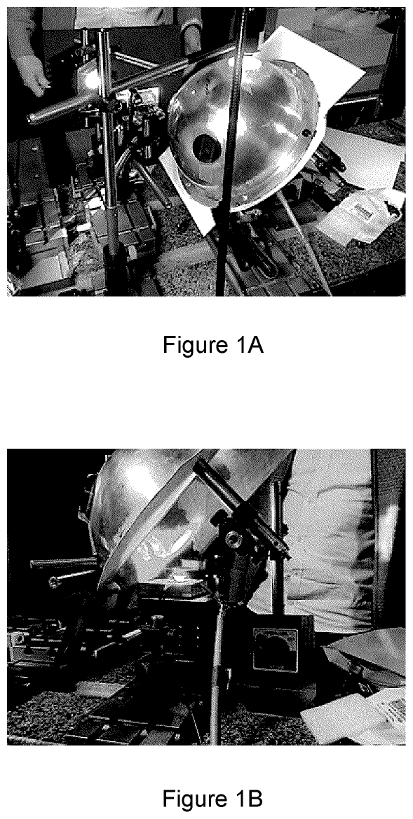

A S-LED light setup was designed and constructed to mimic the energy levels and emission spectra of a fluorescence (primarily in the range of 520 nm and above) emitted from a biophotonic composition comprising a light-absorbing molecule (i.e., Eosin Y), wherein the biophotonic composition is illuminated with light from a multi-LED blue light lamp so as to activate the light-absorbing molecule of the biophotonic composition (due to the light-absorbing molecules absorbance of the incident blue light). The S-LED light setup is shown in FIG. 1A and FIG. 1B.

The S-LED was designed so that a test sample (e.g., a cell or tissue culture sample) could be illuminated by orange and/or blue light. The blue light was emitted from a single blue LED and the orange light was spectrally filtered light from a cold white LED. The spectral filtering was performed using short and long pass dichroic filters where the spectral cut-off wavelength could be tuned by changing the angle of incidence. A half-integrating sphere was produced specially for this S-LED setup, and was used to spatially and spectrally average the spectrally filtered light from the cold white LED on the output port. The output port is 50 mm in diameter. Collimated blue light is directed through the integrating sphere and illuminates the output port directly. The light of the S-LED was produced by using a cold white LED that has a high flux in the pertinent spectral region (around 550 nm) and then the spectral distribution is cut with two dichroic filters (FIG. 1A and FIG. 1B).

### Example 2

### Fluorescence Emitted from an Induced Biophotonic Composition vs. Light Emitted from the S-LED Lamp, Comparison of Modulation of a Biological Process of the Two Types of Light

The scope of the experiment was to examine, on a comparative basis, whether fluorescence generated and emitted by an excited biophotonic composition (“BPC-A”) (in the present Example, a photoconverter gel comprising, in part, Eosin Y as a light-absorbing molecule) may be able to modulate or have an effect on a biological process (measured by total collagen synthesis in a target cell population, in this Example, cultured Dermal Human Fibroblasts) versus whether a light (i.e., a non-fluorescent light) emitted by a multi-LED light system (the S-LED referred to in Example 1) may be able to have a modulatory effect on the same biological process in such cells.

For the present Example, the biophotonic composition was illuminated using a multi-LED blue light lamp (B-LED) (such lamp being referred to as the “B-LED”) positioned at a distance of 5 cm from the photoconverter gel that had been applied to the coverslip (see below for further details of the protocol), having a wavelength range and energetic capacity to induce excitation of the one or light-absorbing molecule due to the light-absorbing molecules' absorption of at least a portion of the light with which they are illuminated. The data obtained was used to make a comparison between any modulatory effects that the fluorescence from the induced biophotonic composition may have had versus the light emitted from the S-LED light. A comparison was also made with respect to the aforementioned treatments to DHF cells that had only been illuminated with the blue light from the B-LED.

The biophotonic system was composed of a photoconverter gel used in combination with a multi-LED lamp emitting blue light (the B-LED). The photoconverter gel was composed of two fractions, namely: a carrier gel comprising, inter alia, a carbopol and urea peroxide, and a light-absorbing molecule gel comprising, inter alia, the light-absorbing molecule Eosin Y. These two gel fractions were freshly mixed before use in 10:1 ratio to obtain a homogenous blend (i.e., the photoconverter gel).

The lamp referred to as B-LED is composed of three panels, each panel containing an identical number of LEDs emitting blue light.

(i) Spectral Comparison

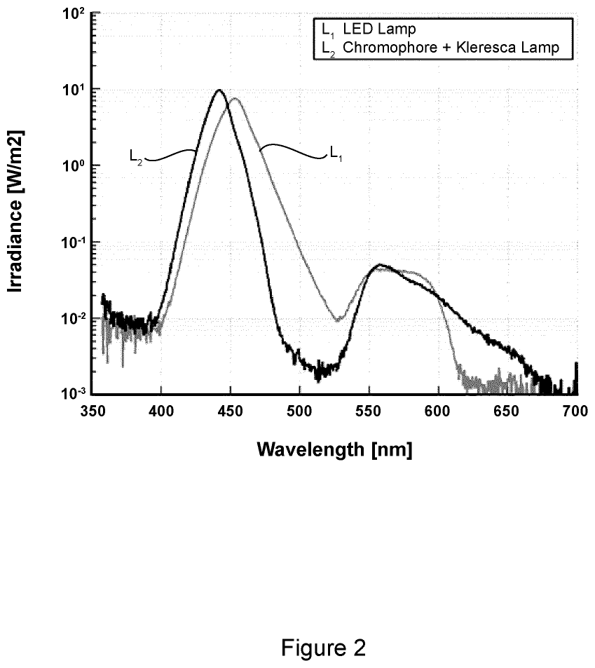

The overlapping spectra of the fluorescence emitted by the biophotonic composition that had been illuminated using the B-LED light and versus the light emitted from the S-LED lamp are presented in FIG. 2. The difference between the two systems is the nature of the light over (i.e., beyond) 520 nm. In the biophotonic system longer wavelengths correspond to the fluorescence emission due the illumination of the light-absorbing molecules.

(ii) Treatment of Dermal Human Fibroblast Cultures

Dermal Human Fibroblasts (DHF) (ATCC, USA) were used as in vitro model to study the effect on production and secretion of collagen proteins by the DHF cells due to the fluorescent light emitted from the induced biophotonic composition and the effect of the non-fluorescent light from the S-LED lamp. Cells were cultured in glass bottom chamber slide (Lab-Tek II, Nalgene Nunc, Denmark) until they reach 60-70% confluency. In some samples Interferon gamma (IFNγ, human recombinant, R&D System, USA) was added to activate the inflammatory pathways. Just before treatment, the cultured medium was replaced for fresh medium.

For the treatment of the DHF cells with the fluorescent light emitted from the induced biophotonic composition, a 2 mm thick layer of the photoconverter gel was applied on the other side of the glass slide and illuminated for 9 min with the B-LED lamp emitting blue light placed at 5 cm distance. There was no direct contact between the photoconverter gel and the cells. Any effect observed was therefore only caused by the transmitted blue light and emitting fluorescence from the photoconverter gel.

In a separate (independent) experiment, total collagen production by DHF cells that were exposed to the only the blue light from the B-LED lamp was evaluated; in this separate experiment, in order to block any thermal effect on the DHFs due to their being illuminated with the B-LED lamp, a 2 mm thickness of a gel equivalent in composition to BPC-A but lacking the light-absorbing molecule was situated on the opposite side of the coverslip to that upon which the cells were cultured (i.e. the gel was not in direct contact with the DHF cells) so as to be between the cells and the light; the gel, however, allowed for the transmittal of the blue light through it in order to illuminate the DHF cells.

After illumination, the photoconverter gel was removed (e.g., washed off from the slide) and the cells were incubated for 48 hours. The dose (expressed in J/cm²) of blue light and fluorescence received by the cells during the treatment period using the photoconverter gel in combination with blue light emitted from the B-LED lamp is presented in Table 2.

TABLE 2
Dose (J/cm²) of blue light and fluorescence received
by the cells during 9 minutes of treatment using a
photoconverter gel in combination with blue light

| Spectrum | J/cm² |
|---|---|
| Purple | 16.13 |
| Blue | 3.52 |
| Green | 0.11 |
| Yellow | 0.10 |
| Orange | 0.03 |
| Red | 0.01 |
| Total J/cm² | 19.90 |

For the treatment of the DHF cells using the light emitted from the S-LED lamp, another set of slides was illuminated with the light emitted from the S-LED lamp (having the same wavelength pattern and radiant fluency as the fluorescent light emitted from the induced biophotonic composition when illuminated with blue light (as described above)).

After 48 h incubation, the culture supernatants were collected. The total collagen production upon the above-noted treatments was evaluated using Sircol Soluble Collagen Assay (Biocolor, UK). Along with measuring collagen production, the treated DHF cells were also assessed for a cytotoxic effect of the fluorescent light treatment and the S-LED light treatment; this was assessed by measuring Lactate Dehydrogenase (LDH) activity using an LDH cytotoxicity assay (Roche, USA) within the culture supernatant.

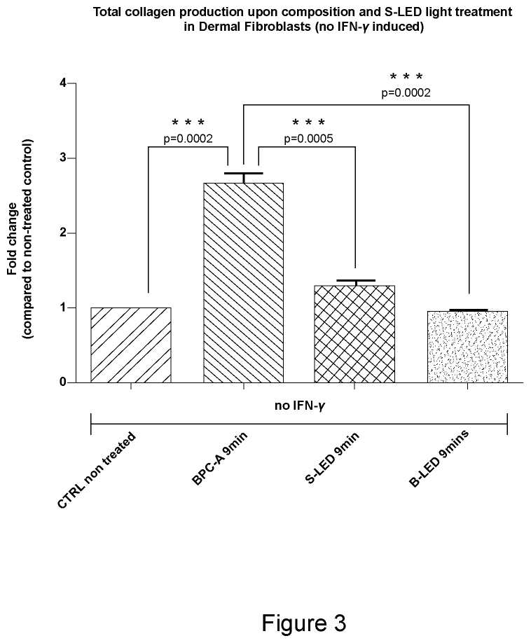

In the first part of the experiment, the DHF cells were left unstimulated (no IFNγ induction). One set of cells was illuminated with the fluorescent light from the biophotonic composition for 9 minutes, at 5 cm distance. The second set of cells was illuminated with the light from the S-LED lamp for 9 min, but the distance from the light source was increased in order to illuminate the entire slide in which cells were cultured. For that reason, the emission spectra of S-LED lamp was not perfectly matching the spectra of the fluorescent light emitted from the induced biophotonic composition. Cells which were not illuminated served as the non-treated control. Close to 3-fold change in collagen synthesis was observed upon the treatment with the fluorescent light emitted from the blue-light illuminated biophotonic composition as compared to non-treated control cells. Interestingly, a 2-fold increase in collagen production was observed in the fibroblasts treated with the fluorescent light emitted from the biophotonic composition system as compared to the cells treated with the light emitted from the S-LED lamp. The treatment with the light from the S-LED lamp showed a low effect on collagen production in the DHF cells. The data are presented in FIG. 3.

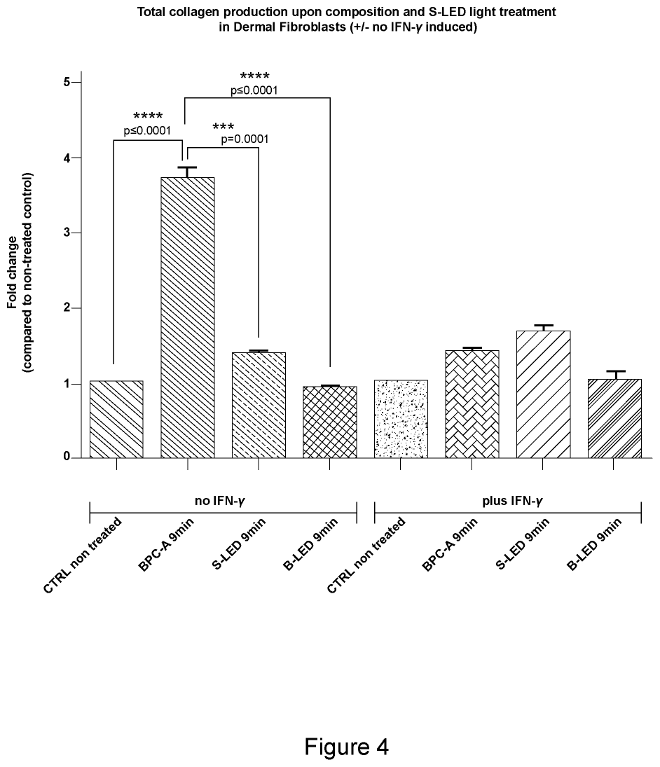

In the second part of the experiment DHF were left unstimulated or stimulated with IFN-γ prior the illumination. IFNγ stimulation served as an inducer of the inflammatory state within the cells. The scope of this set of the experiment was to compare the effect of the inflammation on the collagen synthesis upon an exposure of the DHF cells with either the fluorescent light biophotonic system or multi-LED lamp. Illumination was performed during 9 minutes at 5 cm distance from the light source. Cells not illuminated (+/−IFNγ) served as the non-treated control. The biophotonic treatment induced around 3.5-fold change in collagen production when compared to non-treated control when the cells were not stimulated with IFNγ (normal cells). The treatment of the cells with the multi-LED lamp showed low induction of collagen synthesis when compared to the control. The data are presented in FIG. 4.

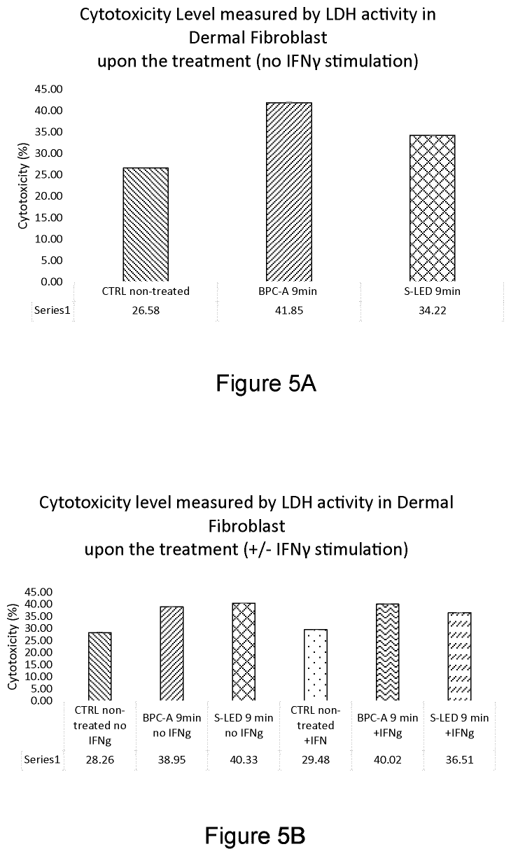

In order to validate the findings of the performed experiments, the cytotoxicity induced within the treated DHF cells was evaluated by measuring the LDH activity in the culture supernatant. The results of the assay are presented in FIGS. 5A-5C.

Overall, the data presented herein suggests that treatment with the fluorescence generated by the blue-light illuminated and induced biophotonic composition significantly up-regulated collagen production in Dermal Human Fibroblasts. Interestingly, the effect of such fluorescence showed a more profound effect on the collagen synthesis compared to the non-fluorescent light generated by the S-LED lamp.

### Example 3

### Ability of Supernatant Derived from Illuminated-Treated Cells to Modulate a Biological Process in Unstimulated Dermal Human Fibroblasts

The scope of the experiment was to evaluate the effects of a group of cells exposed to a photoactivated biophotonic composition of the present technology on another group of cells that are not exposed to a photoactivated biophotonic composition (BPC-A (Eosin Y) or BPC-B (Eosin Y and Fluorescein)) by a multi-LED lamp (biophotonic system).

Dermal human fibroblasts (DHF) were purchased from ATCC (ATCC #PCS-201-012). Cells were thawed and harvested when at 80-90% confluency using Trypsin-EDTA solution for primary cells (ATCC #PCS-999-003) and washed in Trypsin Neutralizing Solution (ATCC #PCS-999-004), counted and seeded in chamber slides with glass bottom (LabTeck, 154852, ThermoFisher) with a density of 80,000 cells/well (for 2 chamber slides). The following day fibroblasts were at 80% confluency and ready to be treated.

Human macrophages were differentiated from monocytic cells, which were positively isolated from human PBMC using CD14 magnetic beads (MACS Miltenyi separation system). Macrophages were differentiated in glass bottom 2 chamber slides for 7 days using GM-CSF at concentration 100 ng/ml. Medium was replaced for fresh one (containing fresh GM-CSF) every three days. Following seven days of differentiation macrophages were fully differentiated, ready to be used in subsequent applications.

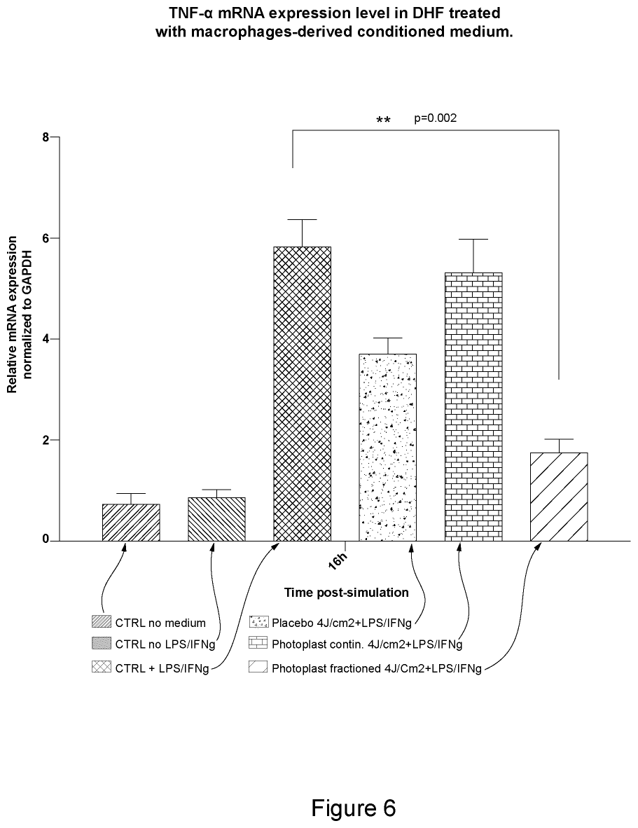

Conditioned culture supernatant derived from formulation-treated macrophages (which were stimulated with LPS/IFNγ and then photoactivated by illumination with the biophotonic system as defined herein) was applied on unstimulated DHF and several genes were screened. The indirect effect of the formulation treatment on fibroblast gene expression profile was evaluated. RNA was isolated at 16 h post-treatment and cells were lysed in RLT Plus (Qiagen) lysis buffer. cDNA was generated and subsequently used in gene expression study. The results of DHF gene expression profile analysis are summarized in FIG. 6.

The results show that that conditioned culture supernatant derived from illuminated-treated macrophages possess the ability to modulate gene expression level in unstimulated dermal human fibroblasts.

### Example 4

### Effects of Fluorescence Emitted from an Induced Biophotonic Composition on Angiogenesis and Tube Formation Process in Human Endothelial Cells

Angiogenesis or neovascularization is the process of generating new blood vessels derived as extensions from the existing vasculature. The principal cells involved in this process are endothelial cells, which line all blood vessels and constitute virtually the entirety of capillaries. Angiogenesis involves multiple steps; to achieve new blood vessel formation, endothelial cells must first escape from their stable location by breaking through the basement membrane. Once this is achieved, endothelial cells migrate toward an angiogenic stimulus such as might be released from keratinocytes, fibroblasts or wound-associated macrophages. In addition, endothelial cells proliferate to provide the necessary number of cells for making a new vessel. Subsequent to this proliferation, the new outgrowth of endothelial cells needs to reorganize into a three-dimensionally tubular structure. Each of these elements, basement membrane disruption, cell migration, cell proliferation, and tube formation, can be a target for intervention, and each can be tested in vitro and in vivo. Several in vivo assay systems, including the chick chorioallantoic membrane (CAM) assay, an in vivo Matrigel plug assay, and the corneal angiogenesis assay, have been developed that permit a more realistic appraisal of the angiogenic response. One quick assessment of angiogenesis is the measurement of the ability of endothelial cells to form three-dimensional structures (tube formation). Endothelial cells retain the ability to divide and migrate rapidly in response to angiogenic signals. Further, endothelial cells are induced to differentiate and form tube-like structures when cultured on matrix of basement membrane extract. These tubes contain a lumen surrounded by endothelial cells linked together through junctional complexes.

Tube formation occurs quickly with most tubes forming in this assay within 2-6 h depending on quantity and type of angiogenic stimuli. Once formed, these interconnected networks are usually maintained for approximately 24 h. Following staining the tube with fluorescence dye, the extent of tube formation, such as average tube length and branch point, can be quantified through microscope connected to imaging software.

Human Aortic Endothelial Cells (HAEC) were used in order to evaluate the potential of conditioned media, derived from Dermal Human Fibroblasts (DHF) treated with a biophotonic composition comprising both Eosin Y and Fluorescein as light-absorbing molecules (BPC-B) according to the present technology on new tubes formation process.

To this end, a 96 wells plate was coated with ice cold matrigel (50 μl/well) and incubated at 37° C. for 45 min to allow the gel to solidify. Next cells were seeded (0.3×105/well) and incubated 1 h at 37° C. in order to adhere to the bottom of matrigel-coated well. Following cell adhesion cell culture medium was replaced by 250 μl of conditioned medium coming from DHF treated with blue light (multi-LED lamp) or BPC-B/Blue light membrane system. The associated control (no treatment) was included as well.

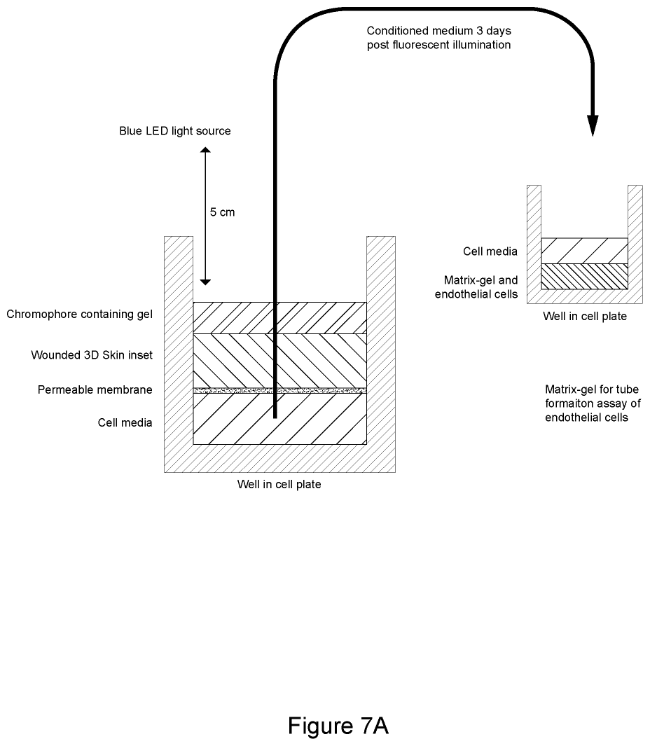

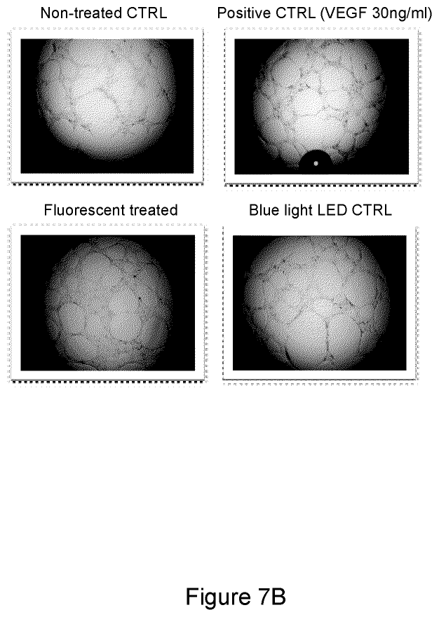

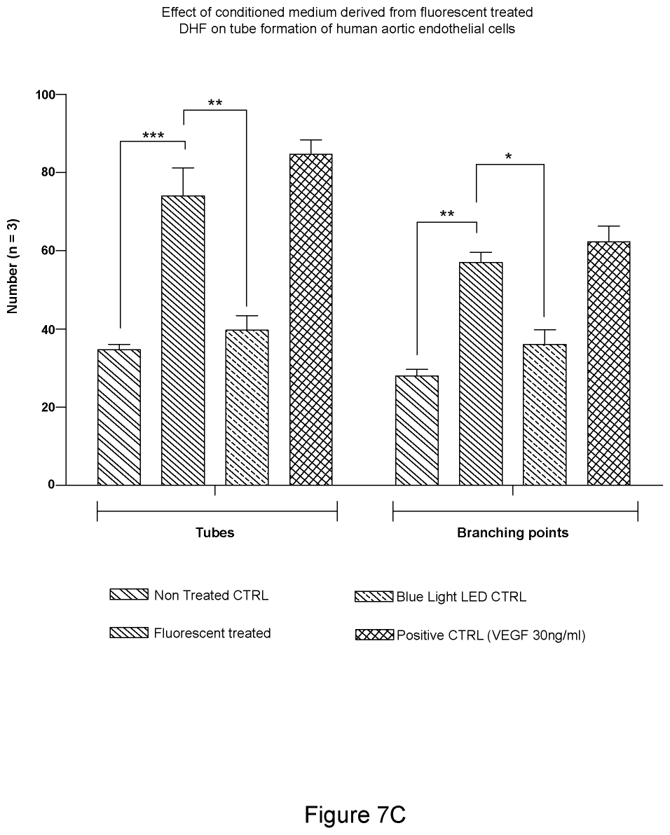

The conditioned media tested were collected at 72 h post treatment with the biophotonic composition and the multi-LED lamp system. Plain medium used for HAEC maintenance was also incorporated as an internal control. Human Aortic Endothelial cells were incubated in the conditioned media for 18 h at 37° C. with 5% CO₂ and tubes network formation was assessed by inverted microscope (Olympus IX50). A schematic representation of the experimental setup is provided in FIG. 7A. Pictures were taken for each well (3 pictures per well) and images were juxtaposed in PowerPoint to quantify the tubes and branching points. The results of the quantification are presented in FIG. 7B and FIG. 7C.

The experimental approach in which conditioned media (obtained from dermal human fibroblasts treated with the multi-LED lamp alone or in the combination with BPC-B composition or BCP-B membrane) was used. Such system allowed to evaluate the potential and activity of conditioned media to induce angiogenesis and tube formation by endothelial cells.

Obtained data proved that conditioned media derived from fibroblast treated with BPC-B composition of BCP-B membrane system provides active growth factors which triggered angiogenesis, which was confirmed by the formation of three-dimensional tube like structures by endothelial cells. Additional analysis of the conditioned media by protein arrays revealed that many pro-angiogenic factors (such as VEGF, ANG, EGF, and TGFβ-1) favouring angiogenesis and new tube formation was secreted by treated dermal fibroblasts. These growth factors retained their activity and acted on endothelial cells, triggering the division, migration and formation of tube-like structures.

Conditioned media derived from cells treated with the multi-LED lamp only or untreated control samples did not induce new tube formation to such extent as has been observed for BPC-B composition of BCB-B membrane. The number of the tubes and branching points was significantly lower.

Interestingly, the branching points and their relative thickness and size formed by endothelial cells cultured in BPC-B composition or BCP-B membrane conditioned medium were increased as compared to control- and multi-LED lamp light-derived conditioned media treated endothelial cells.

In conclusion, a stimulating effect of a biophotonic composition comprising light-absorbing molecules illuminated or photoactivated by a multi-LED lamp on endothelial cells was observed, which proved that the BPC-B composition or BCP-B membrane treatment possess the ability to induce growth factor production in dermal fibroblasts, and that secreted growth factors are biologically active, thus stimulation of angiogenic processes in other cell type (i.e. endothelial cells) was detected.

1.-14. (canceled)

15. A method for modulation of a biological process in a cell or in a tissue, the method comprising:
a) exposing the cell or the tissue to a biophotonic composition or system; and
b) inducing emission of a fluorescence from the biophotonic composition or the system, wherein the fluorescence has spectral emission properties suitable to modulate the biological process;
wherein exposure of the cell or the tissue to the fluorescence modulates the biological process in the cell or the tissue.

16. A method for modulation of a biological process in a cell or in a tissue, the method comprising:
a) photoactivating a biophotonic composition to cause the photoactivated biophotonic composition to emit a fluorescence having spectral emission properties suitable to modulate the biological process; wherein the photoactivation is achieved by exposing the biophotonic composition to a LED light source;
b) removing the photoactivated biophotonic composition from exposure to the LED light source; and
c) exposing the cell or the tissue to the fluorescence emitted by the photoactivated biophotonic composition;
wherein exposure of the cell or the tissue to the fluorescence modulates the biological process in the cell or the tissue.

17. The method as defined in claim 15, wherein the modulation is photobiornodulation.

18. The method as defined in claim 15, wherein the cells or the tissue is not exposed artificial source of light.

19. The method as defined in claim 15, wherein the biophotonic composition is not in direct contact with the cell or the tissue.

20. The method as defined in claim 15, wherein the biophotonic composition comprises one or more light-absorbing molecules.

21. The method as defined in claim 20, wherein the one or more light-absorbing molecules emit the fluorescence.

22. The method as defined in claim 20, or wherein the one or more liobt-absorbing molecules are one or more xanthene dyes.

23. The method as defined in claim 22, wherein the one or more xanthene dyes are Eosin.

24. The method as defined in claim 23, wherein the Eosin is Eosin Y.

25. The method as defined in claim 22, wherein the one or more light-absorbing molecules are Eosin Y and Fluorescein.

26. The method as defined in claim 15, wherein the biological process is a cellular process.

27. The method as defined in claim 26, wherein the cellular process is a signaling pathway.

28. The method as defined in claim 15, wherein the biological process is an inflammatory response pathway.

29. The method defined in claim 15, wherein the biological process is a collagen formation pathway.

30. The method as defined in claim 15, wherein the biological process is angiogenesis.

31.-32. (canceled)
